(19) United States
(12) Patent Application Publication
(10) Pub. No.: US 2025/0186220 A1
(43) Pub. Date: Jun. 12, 2025

(54) SACROILIAC FUSION SCREW

(71) Applicant: HT MEDICAL, LLC, Tulsa, OK (US)

(72) Inventors: Tyler HOLSCHLAG, Vista, CA (US); Todd ABEL, Knoxville, TN (US); Joseph Christopher WEHMAN, Windermere, FL (US); Joshua MILLER, Knoxville, TN (US)

(21) Appl. No.: 18/978,025

(22) Filed: Dec. 12, 2024

Related U.S. Application Data
(60) Provisional application No. 63/609,273, filed on Dec. 12, 2023.

(51) Int. Cl. A61F 2/44; A61F 2/30

(52) U.S. Cl. CPC A61F 2/446; A61F 2/30771; A61F 2002/30593; A61F 2002/30851; A61F 2002/3093

(57) ABSTRACT

In some embodiments, an implant configured to span a joint between a first bone and a second bone may include a proximal end configured to reside in the first bone, a distal end including threads configured to reside in the second bone distal to the first bone in response to rotation of the implant about an axis of rotation, an axis extending between the proximal end and the distal end, and an exterior surface extending from the proximal end to the distal end. The exterior surface may include a first recess formed therein. The first recess may include an undercut such that the first recess has a first perimeter at an exterior of the first recess, and a second perimeter, greater than the first perimeter, at an interior of the first recess, wherein the interior is closer to the axis than the exterior.

### CROSS-REFERENCE TO RELATED APPLICATIONS

The present application claims the benefit of U.S. Provisional Patent Application Ser. No. 63/609,273, filed on Dec. 12, 2023, entitled SACROILIAC FUSION SCREW, which is incorporated by reference as though set forth herein in its entirety.

### TECHNICAL FIELD

The present disclosure relates to systems and methods for use in orthopedic surgery. More specifically, the present disclosure relates to sacroiliac fusion screw systems and surgical methods.

### BACKGROUND

The sacroiliac joint, formed between the sacrum and ilium bones of the pelvis, is crucial for load transmission between the spine and the lower extremities. Subsequently, dysfunction of this joint can lead to debilitating pain and instability, often requiring surgical intervention for stabilization and pain relief. Current techniques for sacroiliac joint fusion typically involve the use of screws and other implants to create stability and promote fusion between the sacrum and ilium. However, existing devices have limitations such as inadequate fixation strength, difficulty in implantation, or insufficient bone ingrowth. Furthermore, existing screws may not always provide sufficient initial fixation or long-term stability, leading to potential complications such as implant loosening or migration.

### SUMMARY

The various systems and methods of the present disclosure have been developed in response to the present state of the art, and in particular, in response to the problems and needs in the art that have not yet been fully solved by currently available sacroiliac fusion screw systems.

In some embodiments, an implant configured to span a joint between a first bone and a second bone may include a proximal end configured to reside in the first bone, a distal end including threads configured to reside in the second bone distal to the first bone in response to rotation of the implant about an axis of rotation, an axis extending between the proximal end and the distal end, and an exterior surface extending from the proximal end to the distal end. The exterior surface may include a first recess formed therein. The first recess may include an undercut such that the first recess has a first perimeter at an exterior of the first recess, and a second perimeter, greater than the first perimeter, at an interior of the first recess, wherein the interior is closer to the axis than the exterior.

In the implant of any preceding paragraph, the implant may further include an inner cannula extending from the proximal end to the distal end. The inner cannula may include a first diameter at the proximal end and the distal end and a second diameter in an intermediate portion between the proximal end and the distal end.

In the implant of any preceding paragraph, the first diameter may be configured to receive a guidewire with minimal clearance to facilitate placement of the implant over the guidewire. The second diameter may be configured to receive a bone graft material and may be larger than the first diameter.

In the implant of any preceding paragraph, the exterior surface may include a second recess. The second recess may include a second undercut such that the second recess has a third perimeter at an exterior of the second recess and a fourth perimeter, greater than the third perimeter, at an interior of the second recess. A second geometric shape of the second recess may be different than a first geometric shape of the first recess.

In the implant of any preceding paragraph, the first geometric shape of the first recess may be non-circular and the second geometric shape of the second recess may be non-circular.

In the implant of any preceding paragraph, the third perimeter may be less than the first perimeter.

In the implant of any preceding paragraph, the first recess may not intersect with the second recess.

In the implant of any preceding paragraph, the implant may further include an inner cannula extending from the proximal end to the distal end, and the first recess may further include a first recess depth between the exterior of the first recess and the interior of the first recess. The first recess depth may be less than a radial distance between the inner cannula and the exterior surface.

In some embodiments, an implant configured to span a joint between a first bone and a second bone may include a proximal end configured to reside in the first bone, a distal end including threads configured to reside in the second bone distal to the first bone in response to rotation of the implant about an axis of rotation, and an inner cannula extending from the proximal end to the distal end. The inner cannula may include a first diameter at the proximal end and the distal end and a second diameter in an intermediate portion between the proximal end and the distal end. The first diameter may be configured to receive a guidewire with minimal clearance to facilitate placement of the implant over the guidewire, and the second diameter may be configured to receive a bone graft material and may be larger than the first diameter.

In the implant of any preceding paragraph, the implant may further include an exterior surface extending from the proximal end to the distal end. The exterior surface may include a first recess formed therein. The first recess may include an undercut such that the first recess has a first perimeter at an exterior of the first recess and a second perimeter, greater than the first perimeter, at an interior of the first recess.

In the implant of any preceding paragraph, the first recess may include a first recess depth between the exterior of the first recess and the interior of the first recess. The first recess depth may be less than a radial distance between the inner cannula and the exterior surface.

In the implant of any preceding paragraph, the exterior surface may further include a second recess. The second recess may include a second undercut such that the second recess has a third perimeter at an exterior of the second recess and a fourth perimeter, greater than the third perimeter, at an interior of the second recess. A second geometric shape of the second recess may be different than a first geometric shape of the first recess.

In the implant of any preceding paragraph, the first geometric shape of the first recess may be non-circular and the second geometric shape of the second recess may be non-circular.

In the implant of any preceding paragraph, the third perimeter may be less than the first perimeter.

In the implant of any preceding paragraph, the first recess may not intersect with the second recess.

In some embodiments, an implant configured to span a joint between a first bone and a second bone may include a proximal end configured to reside in the first bone, a distal end including threads configured to reside in the second bone distal to the first bone in response to rotation of the implant about an axis of rotation, an inner cannula extending from the proximal end to the distal end, and an exterior surface extending from the proximal end to the distal end. The exterior surface may include a first recess formed therein. The first recess may include an undercut such that the first recess has a first perimeter at an exterior of the first recess, and a second perimeter, greater than the first perimeter, at an interior of the first recess. The exterior of the first recess may be located on the exterior surface, and the interior of the first recess is located between the exterior surface and the inner cannula.

In the implant of any preceding paragraph, the implant may further include an inner cannula extending from the proximal end to the distal end. The inner cannula may include a first diameter at the proximal end and the distal end and a second diameter in an intermediate portion between the proximal end and the distal end.

In the implant of any preceding paragraph, the first diameter may be configured to receive a guidewire with minimal clearance to facilitate placement of the implant over the guidewire, and the second diameter may be configured to receive a bone graft material and may be larger than the first diameter.

In the implant of any preceding paragraph, the exterior surface may further include a second recess. The second recess may include a second undercut such that the second recess has a third perimeter at an exterior of the second recess and a fourth perimeter, greater than the third perimeter, at an interior of the second recess. A second geometric shape of the second recess is different that a first geometric shape of the first recess.

In the implant of any preceding paragraph, the first recess may not intersect with the second recess.

These and other features and advantages of the present disclosure will become more fully apparent from the following description taken in conjunction with the accompanying drawings. Understanding that these drawings depict only exemplary embodiments and are, therefore, not to be considered limiting of the scope of the present disclosure, the exemplary embodiments of the present disclosure will be described with additional specificity and detail through the use of accompanying drawings.

### BRIEF DESCRIPTION OF THE DRAWINGS

Exemplary embodiments of the disclosure will become more fully apparent from the following description and appended claims, taken in conjunction with the accompanying drawings. Understanding that these drawings depict only exemplary embodiments and are, therefore, not to be considered limiting of the scope of the appended claims, the exemplary embodiments of the disclosure will be described with additional specificity and detail through use of the accompanying drawings in which:

FIG. 1 is a perspective view of a sacroiliac fusion screw according to an embodiment;

FIG. 2 is a front view of the sacroiliac fusion screw of FIG. 1;

FIG. 3 is a right-side view of the sacroiliac fusion screw of FIG. 1;

FIG. 4 is a left-side view of the sacroiliac fusion screw of FIG. 1;

FIG. 5 is a front section view of the sacroiliac fusion screw of FIG. 1;

FIG. 6 is a partial front section view of the sacroiliac fusion screw of FIG. 1;

FIG. 7 is a front view of the sacroiliac fusion screw of FIG. 1 coupled to an inserter assembly according to an embodiment;

FIG. 8 is a partial front view of the sacroiliac fusion screw coupled to the inserter assembly of FIG. 7.

### DETAILED DESCRIPTION

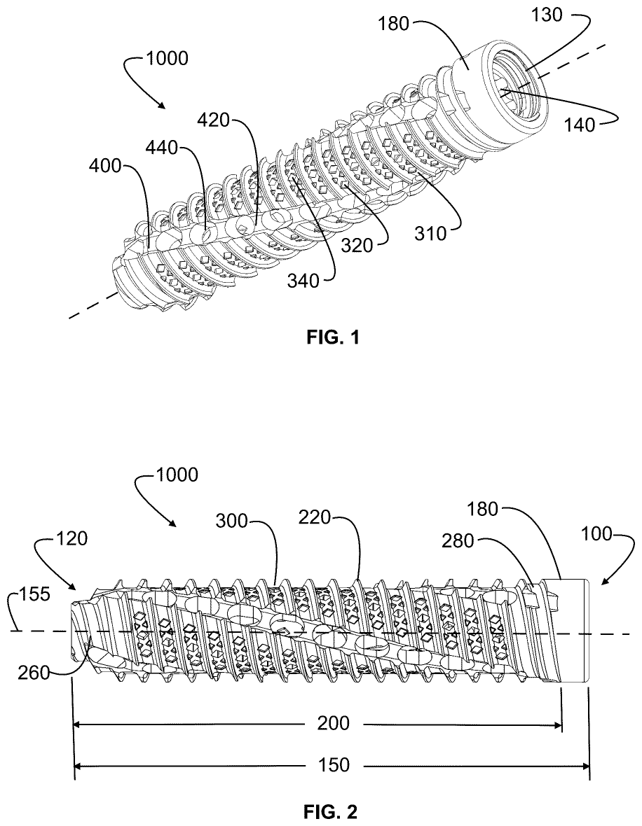

Exemplary embodiments of the disclosure will be best understood by reference to the drawings, wherein like parts are designated by like numerals throughout. It will be readily understood that the components of the disclosure, as generally described and illustrated in the Figures herein, could be arranged and designed in a wide variety of different configurations. Thus, the following more detailed description of the embodiments of the apparatus, system, and method, as represented in FIG. 1 through FIG. 8, is not intended to limit the scope of the claims, but is merely representative of exemplary embodiments of the present disclosure.

The phrases “connected to,” “coupled to” and “in communication with” refer to any form of interaction between two or more entities, including mechanical, electrical, magnetic, electromagnetic, fluid, and thermal interaction. Two components may be functionally coupled to each other even though they are not in direct contact with each other. The term “abutting” refers to items that are in direct physical contact with each other, although the items may not necessarily be attached together.

The word “exemplary” is used herein to mean “serving as an example, instance, or illustration.” Any embodiment described herein as “exemplary” is not necessarily to be construed as preferred or advantageous over other embodiments. While the various aspects of the embodiments are presented in drawings, the drawings are not necessarily drawn to scale unless specifically indicated.

Not every feature of each embodiment is labeled in every figure in which that embodiment appears, in order to keep the figures clear. Similar reference numbers (for example, those that are identical except for the first numeral) may be used to indicate similar features in different embodiments.

The present disclosure illustrates devices for sacroiliac (SI) joint fusion for the purposes of illustrating the concepts of the present design. However, it will be understood that other variations and uses are contemplated including, but not limited to, devices for facet fixation of adjacent vertebra, etc.

FIG. 1 is a perspective view of a sacroiliac fusion screw 1000 according to an embodiment. FIG. 2 is a front view of the sacroiliac fusion screw 1000. In an embodiment, the sacroiliac fusion screw 1000 may be configured to span a joint between two bones. Additionally, or alternatively, the sacroiliac fusion screw 1000 may be configured to augment stabilization and immobilization of the sacroiliac joint. The sacroiliac fusion screw 1000 may be configured to facilitate fusion between two adjacent bones of a joint.

The sacroiliac fusion screw 1000 may have a proximal end 100 and a distal end 120. The proximal end may be configured to reside in a first bone and may have a connector portion 130 and a drive portion 140. The drive portion 140 may be configured to facilitate rotation of the sacroiliac fusion screw 1000 about an axis of rotation 155 through engagement with a drive shaft 2200. The axis of rotation 155 may extend between the proximal end 100 and the distal end 120. The drive portion 140 may be configured as a hex, a hexalobe, a square or other non-circular profile known in the art. The connector portion 130 may be configured to receive a connector shaft 2300 configured to retain the sacroiliac fusion screw 1000 on the drive shaft 2200. The connector portion 130 may be configured as a female thread, generally concentric to the axis of rotation 155. Alternatively, the connector portion 130 may be configured as a male thread, generally concentric to the axis of rotation 155. Alternatively, the connector portion 130 may be configured with a removeable engagement feature configured to retain the sacroiliac fusion screw 1000 on the drive shaft 2200.

The sacroiliac fusion screw 1000 may have a length 150 extending from the proximal end 100 to the distal end 120. The length 150 may be within a range of lengths from 20 mm to 90 mm. In an embodiment, the sacroiliac fusion screw 1000 may be one of a set of differently-sized implants, each having a different length. The sacroiliac fusion screw 1000 may include a threaded portion 200 configured to engage a first bone and a second bone. The threaded portion 200 may have a major diameter 220 and a minor diameter 300. The major diameter 220 may be within a range of diameters from 7 mm to 15 mm. In an embodiment, the sacroiliac fusion screw 1000 may be one of a set of differently-sized implants, each having a different diameter.

The distal end 120 may be configured to reside in a second bone distal to the first bone in response to rotation of the implant about an axis of rotation 155. The distal end 120 may have a thread lead-in portion 260 configured to facilitate engagement of the threaded portion 200 with the first bone. The thread lead-in portion may be configured as a tapered thread such that the thread diameter at the distal end 120 may be less than the major diameter 220 of the sacroiliac fusion screw 1000.

The proximal end 100 may include a screw head 180 configured as an exterior portion of the sacroiliac fusion screw 1000 between the end of the threaded portion 200 and the proximal end 100. The screw head 180 may have generally the same diameter as the major diameter 220. Alternatively, the screw head 180 may have a diameter that is larger than the major diameter 220. Alternatively, the screw head 180 may have a diameter that is smaller than the major diameter 220. The threaded portion 200 may have a thread run-out portion 280 configured as a transitional threaded portion wherein the thread tapers from the minor diameter 300 to the screw head 180.

The minor diameter 300 may include an exterior surface 310 extending from the proximal end 100 to the distal end 120. The exterior surface 310 may have a plurality of first recesses 320 and a plurality of second recesses 340 to facilitate bone ingrowth, minimize rotation after implantation, and/or reduce implant migration. The plurality of first recesses 320 may be interspersed with the plurality of second recesses 340 in an alternating pattern along the exterior surface 310. Each of the plurality of first recesses 320 may be configured as a shape from one or more of: a diamond, a triangle, a circle, an oval, a square, a pentagon, a hexagon, an octagon, a trapezoid, a parallelogram, a rectangle, or other geometric shape.

Each of the plurality of second recesses 340 may be configured as a shape from one or more of: a diamond, a circle, an oval, a square, a triangle, a pentagon, a hexagon, an octagon, a trapezoid, a parallelogram, a rectangle, or other geometric shape. Each of the plurality of first recesses 320 may not intersect with each of the plurality of second recesses 340.

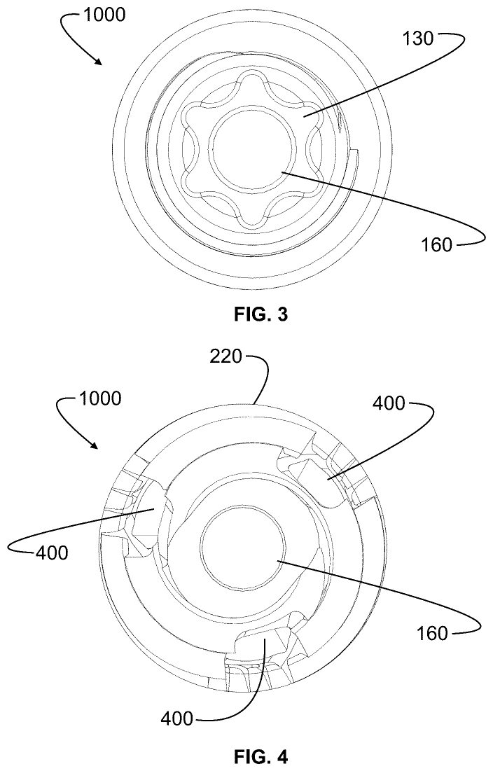

FIG. 3 is a right-side view of the sacroiliac fusion screw 1000 and FIG. 4 is a left-side view of the sacroiliac fusion screw 1000. The sacroiliac fusion screw 1000 may have one or more flutes 400 configured to facilitate bone in-growth, minimize rotation after implantation, and reduce implant migration. The one or more flutes 400 may further be configured as tapping flutes wherein the flutes 400 facilitate engaging the threaded portion 200 with a bone and provide a channel for removal of any bone debris resulting from cutting the thread form profile into the bone.

The one or more flutes 400 may be configured to interrupt the major diameter 220 along the threaded portion 200. The one or more flutes 400 may also be deep enough to interrupt the minor diameter 300 of the threaded portion 200. The one or more flutes 400 may extend along the length of the threaded portion 200 in a helical manner. Alternatively, the one or more flutes 400 may extend along the length of the threaded portion 200 in a straight manner.

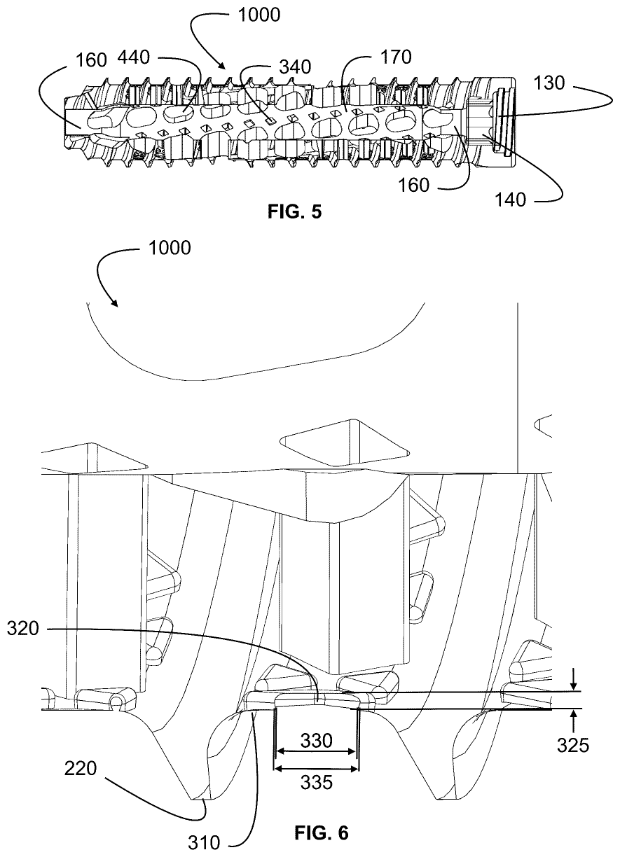

FIG. 5 is a front section view of the sacroiliac fusion screw 1000 and FIG. 6 is a partial front section view of the sacroiliac fusion screw 1000. The sacroiliac fusion screw 1000 may have a first inside diameter 160 and a second inside diameter 170 defining a non-uniform inner cannula to allow for in-site graft delivery and promote bone ingrowth through the sacroiliac fusion screw 1000. The first inside diameter 160 may be configured to slidably receive a guidewire during deployment of the sacroiliac fusion screw 1000 into a bone. The first diameter may be sized to receive the guidewire with minimal clearance. The second inside diameter 170 may be configured to receive bone graft material or other suitable materials that are known in the art to promote fusion of adjacent bones. The second inside diameter 170 may be larger than the first inside diameter 160 to allow for increased bone graft volume.

Alternatively, the first inside diameter 160 may be generally equal to the second inside diameter 170. The first inside diameter 160 may be present at the proximal end 100 and the distal end 120 to allow a guidewire to pass through the distal cannulation and then guide back into the proximal cannulation to exit the implant. The inner cannula may include an intermediate portion between proximal end 100 and the distal end 120. The intermediate portion may include the second inside diameter 170. Thus, an enlarged graft volume may be used, without requiring a larger guidewire or sacrificing accuracy in placement of the sacroiliac fusion screw 1000.

The one or more flutes 400 each may have a flute surface 420. Each flute surface 420 may have a plurality of flute apertures 440. The plurality of flute apertures 440 may have a width generally equal to the width of the flute 400. The plurality of flute apertures 440 may be configured as a shape from one or more of: an oval, a diamond, a circle, a square, a triangle, a pentagon, a hexagon, an octagon, a trapezoid, a parallelogram, a rectangle, or other geometric shape. The plurality of flute apertures 440 may extend from the flute surface 420 to the second inside diameter 170 to permit growth of a column of bone between the flute surface 420 and the second inside diameter 170.

The plurality of second recesses 340 along an exterior surface 310 may extend from the exterior surface 310 to the second inside diameter 170 to permit growth of a column of bone between the exterior surface 310 and the second inside diameter 170.

The plurality of first recesses 320 may configured as a plurality of purposeful repeated geometric debossing (or recesses). Each of the plurality of first recesses 320 may have a first recess depth 325 wherein the first recess depth 325 may be less than the radial distance between the second inside diameter 170 and the exterior surface 310. Additionally, each of the plurality of first recesses 320 may have a first perimeter 330 and a second perimeter 335 wherein the first perimeter 330 may be located at the exterior surface 310. The second perimeter may be located at an interior of the first recess 320 at a depth equal to the first recess depth 325. Each of the plurality of first recesses 320 may be an undercut to enhance engagement of bone in-growth, wherein the second perimeter 335 may be greater than the first perimeter 330. The interior of the first recess 320 may be closer to the axis of rotation 155 than the exterior surface 310.

When applied to a recess on an object, the term “interior” refers to a portion of the recess that is relatively closer to the axis or center of the object, and the term “exterior” refers to a portion of the recess that is relatively further from the axis or center. In some embodiments, an interior of a recess may be within the recess, while the exterior of the recess may be at an aperture that provides entry into the recess. An interior of a recess may be at a blind wall, an interior opening leading to a hollow interior such as a cannulation, or a point exteriorly displaced from a blind wall or interior opening.

The first perimeter 330 may equal a length of a continuous line forming a boundary of a closed geometric figure located at the exterior surface 310. The second perimeter 335 may equal a length of a continuous line forming a boundary of a closed geometric figure located at the interior of the first recess 320 at the depth equal to the first recess depth 325.

Alternatively, each of the plurality of first recesses 320 may have a first recess depth 325 wherein the first recess depth 325 may be equal to the radial distance between the second inside diameter 170 and the exterior surface 310. Additionally, each of the plurality of first recesses 320 may have a first perimeter 330 and a second perimeter 335 wherein the first perimeter 330 may be located at the exterior surface 310. The second perimeter may be located at an interior of the first recess 320 at a depth equal to the first recess depth 325. Each of the plurality of first recesses 320 may be an undercut to enhance engagement of bone in-growth, wherein the second perimeter 335 may be greater than the first perimeter 330.

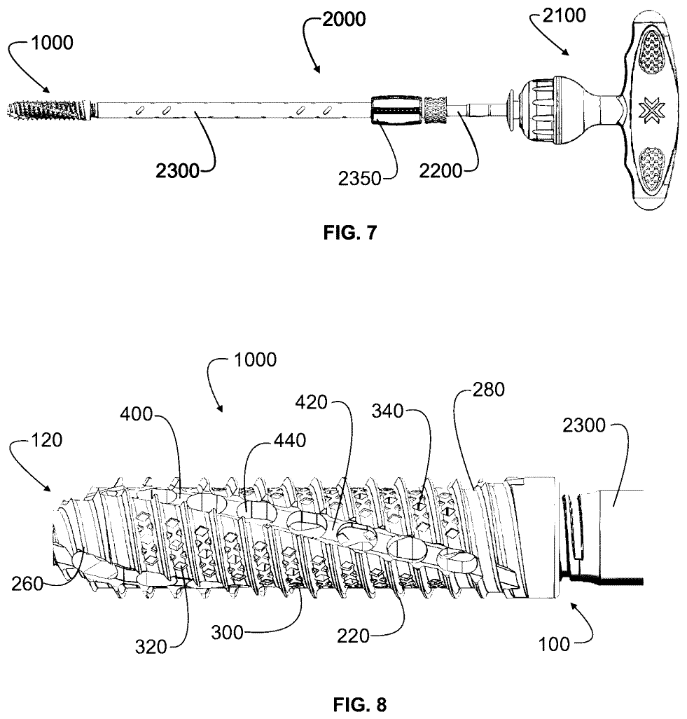

FIG. 7 is a front view of the sacroiliac fusion screw 1000 coupled to an inserter assembly 2000 according to an embodiment. FIG. 8 is a partial front view of the sacroiliac fusion screw 1000 coupled to the inserter assembly 2000. The inserter assembly 2000 may be configured to securably and removably couple to the sacroiliac fusion screw 1000 to facilitate deployment of the sacroiliac fusion screw 1000 into a bone through rotation of the sacroiliac fusion screw 1000. The inserter assembly may include a handle 2100, a drive shaft 2200, a connector shaft 2300 and a connector handle 2350. The drive shaft 2200 may be configured to engage the drive portion 140 of the sacroiliac fusion screw 1000 wherein rotation of the drive shaft 2200 results in rotation of the sacroiliac fusion screw 1000.

The connector shaft 2300 may be configured to threadably engage the connector portion 130 of the sacroiliac fusion screw 1000, wherein engagement of the connector shaft 2300 with the connector portion 130 may prevent disengagement of the drive shaft 2200 from the drive portion 140. The connector handle 2350 may be configured to rotate the connector shaft 2300 to facilitate engagement and disengagement of the connector shaft 2300 with the connector portion 130. The handle 2100 may be configured to couple with the drive shaft 2200 wherein rotation of the handle 2100 results in rotation of the drive shaft 2200. The connector portion 130 may be configured to threadably receive the connector shaft 2300.

Any of the devices described herein may be fabricated from metals, alloys, polymers, plastics, ceramics, glasses, composite materials, or combinations thereof, including but not limited to titanium, titanium alloy, stainless steel, PEEK (polyether ether ketone), among others. Different materials may be used within a single part.

Any of the devices described herein may be fabricated utilizing an additive manufacturing process. Additive manufacturing processes may include, but are not limited to, three-dimensional printing (3DP) processes, laser-net-shape manufacturing, direct metal laser sintering (DMLS), direct metal laser melting (DMLM), plasma transferred arc, freeform fabrication, direct digital manufacturing, layered manufacturing, and rapid prototyping.

Reference throughout this specification to “an embodiment” or “the embodiment” means that a particular feature, structure or characteristic described in connection with that embodiment is included in at least one embodiment. Thus, the quoted phrases, or variations thereof, as recited throughout this specification are not necessarily all referring to the same embodiment.

Similarly, it should be appreciated that in the above description of embodiments, various features are sometimes grouped together in a single embodiment, Figure, or description thereof for the purpose of streamlining the disclosure. This method of disclosure, however, is not to be interpreted as reflecting an intention that any claim require more features than those expressly recited in that claim. Rather, as the following claims reflect, inventive aspects lie in a combination of fewer than all features of any single foregoing disclosed embodiment. Thus, the claims following this Detailed Description are hereby expressly incorporated into this Detailed Description, with each claim standing on its own as a separate embodiment. This disclosure includes all permutations of the independent claims with their dependent claims.

The phrases “generally parallel” and “generally perpendicular” refer to structures that are within 30° parallelism or perpendicularity relative to each other, respectively. Recitation in the claims of the term “first” with respect to a feature or element does not necessarily imply the existence of a second or additional such feature or element. Elements recited in means-plus-function format are intended to be construed in accordance with 35 U.S.C. § 112 Para. 6. It will be apparent to those having skill in the art that changes may be made to the details of the above-described embodiments without departing from the underlying principles of the disclosure.

While specific embodiments and applications of the present disclosure have been illustrated and described, it is to be understood that the disclosure is not limited to the precise configuration and components disclosed herein. Various modifications, changes, and variations which will be apparent to those skilled in the art may be made in the arrangement, operation, and details of the methods and systems of the present disclosure without departing from its spirit and scope.

1. An implant configured to span a joint between a first bone and a second bone, the implant comprising:
a proximal end configured to reside in the first bone;
a distal end comprising threads configured to reside in the second bone distal to the first bone in response to rotation of the implant about an axis of rotation;
an axis extending between the proximal end and the distal end; and
an exterior surface extending from the proximal end to the distal end, the exterior surface comprising a first recess formed therein, the first recess comprising an undercut such that the first recess has:
a first perimeter at an exterior of the first recess; and
a second perimeter, greater than the first perimeter, at an interior of the first recess, wherein the interior is closer to the axis than the exterior.

2. The implant of claim 1, further comprising an inner cannula extending from the proximal end to the distal end, the inner cannula comprising a first diameter at the proximal end and the distal end and a second diameter in an intermediate portion between the proximal end and the distal end.

3. The implant of claim 2, wherein the first diameter is configured to receive a guidewire with minimal clearance to facilitate placement of the implant over the guidewire; and the second diameter is configured to receive a bone graft material and is larger than the first diameter.

4. The implant of claim 1, wherein the exterior surface further comprises a second recess, the second recess comprising a second undercut such that the second recess has a third perimeter at a second exterior of the second recess and a fourth perimeter, greater than the third perimeter, at a second interior of the second recess, wherein a second geometric shape of the second recess is different than a first geometric shape of the first recess.

5. The implant of claim 4, wherein the first geometric shape of the first recess is non-circular and the second geometric shape of the second recess is non-circular.

6. The implant of claim 4, wherein the third perimeter is less than the first perimeter.

7. The implant of claim 4, wherein the first recess does not intersect with the second recess.

8. The implant of claim 1, wherein the implant further comprises an inner cannula extending from the proximal end to the distal end, and the first recess further comprises a first recess depth between the exterior of the first recess and the interior of the first recess, wherein the first recess depth is less than a radial distance between the inner cannula and the exterior surface.

9. The implant of claim 1, wherein the implant further comprises an inner cannula extending from the proximal end to the distal end, the interior of the first recess is located at the inner cannula, and the first recess extends from the exterior surface to the inner cannula.

10. An implant configured to span a joint between a first bone and a second bone, the implant comprising:
a proximal end configured to reside in the first bone;
a distal end comprising threads configured to reside in the second bone distal to the first bone in response to rotation of the implant about an axis of rotation; and
an inner cannula extending from the proximal end to the distal end, the inner cannula comprising a first diameter at the proximal end and the distal end and a second diameter in an intermediate portion between the proximal end and the distal end;
wherein:
the first diameter is configured to receive a guidewire with minimal clearance to facilitate placement of the implant over the guidewire; and
the second diameter is configured to receive a bone graft material and is larger than the first diameter.

11. The implant of claim 10, further comprising an axis extending between the proximal end and the distal end, an exterior surface extending from the proximal end to the distal end, the exterior surface comprising a first recess formed therein, the first recess comprising an undercut such that the first recess has a first perimeter at an exterior of the first recess and a second perimeter, greater than the first perimeter, at an interior of the first recess, wherein the interior is closer to the axis than the exterior.

12. The implant of claim 11, wherein the first recess comprises a first recess depth between the exterior of the first recess and the interior of the first recess, wherein the first recess depth is less than a radial distance between the inner cannula and the exterior surface.

13. The implant of claim 11, wherein the exterior surface further comprises a second recess, the second recess comprising a second undercut such that the second recess has a third perimeter at a second exterior of the second recess and a fourth perimeter, greater than the third perimeter, at a second interior of the second recess, wherein a second geometric shape of the second recess is different than a first geometric shape of the first recess.

14. The implant of claim 13, wherein the first geometric shape of the first recess is non-circular and the second geometric shape of the second recess is non-circular.

15. The implant of claim 13, wherein the third perimeter is less than the first perimeter.

16. The implant of claim 13, wherein the first recess does not intersect with the second recess.

17. An implant configured to span a joint between a first bone and a second bone, the implant comprising:
a proximal end configured to reside in the first bone;
a distal end comprising threads configured to reside in the second bone distal to the first bone in response to rotation of the implant about an axis of rotation;
an axis extending between the proximal end and the distal end;
an inner cannula extending from the proximal end to the distal end, and
an exterior surface extending from the proximal end to the distal end, the exterior surface comprising a first recess formed therein, the first recess comprising an undercut such that the first recess has:
a first perimeter at an exterior of the first recess; and
a second perimeter, greater than the first perimeter, at an interior of the first recess;
wherein:
the interior is closer to the axis than the exterior;
the exterior of the first recess is located on the exterior surface; and
the interior of the first recess is located between the exterior surface and the inner cannula.

18. The implant of claim 17, further comprising an inner cannula extending from the proximal end to the distal end, the inner cannula comprising a first diameter at the proximal end and the distal end and a second diameter in an intermediate portion between the proximal end and the distal end, wherein the second diameter is larger than the first diameter.

19. The implant of claim 17, wherein the exterior surface further comprises a second recess, the second recess comprising a second undercut such that the second recess has a third perimeter at a second exterior of the second recess and a fourth perimeter, greater than the third perimeter, at a second interior of the second recess, wherein a second geometric shape of the second recess is different than a first geometric shape of the first recess.

20. The implant of claim 19, wherein the first recess does not intersect with the second recess.
